(19) United States
(12) Patent Application Publication
(10) Pub. No.: US 2019/0275086 A1
(43) Pub. Date: Sep. 12, 2019

(54) METHOD FOR IN-SITU RADIATION-PRIMED T-CELL THERAPY

(71) Applicants: Xiaodong Wu, Miami, FL (US); Sunil Krishnan, Houston, TX (US)

(72) Inventors: Xiaodong Wu, Miami, FL (US); Sunil Krishnan, Houston, TX (US)

(21) Appl. No.: 16/163,542

(22) Filed: Oct. 17, 2018

Related U.S. Application Data
(60) Provisional application No. 62/573,192, filed on Oct. 17, 2017.

(51) Int. Cl. A61K 35/26; A61N 5/10; C12N 5/0783

(52) U.S. Cl. CPC A61K 35/26; C12N 5/0638; A61N 5/10

(57) ABSTRACT

The present invention as described herein is aimed at combining a radiation-induced immunogenic effect with a T-cell therapy technique to markedly improve the therapeutic effectiveness of adoptive T-cell therapy with minimized toxicity. The method of this invention comprises, identifying a target tumor, applying ablative radiation treatment to the tumor in-situ, waiting for the production of CTLs primed by antigen presenting cells (APC), then resecting the target tumor from the patient. The CTLs are harvested and isolated from the tumor and undergo ex-vivo expansion and subsequent treatment of immune checkpoint blockades. The expanded CTLs are then infused back into the patient for systemic treatment of microscopic disease. The primed CTLs that are induced by radiation in-situ, are used as the source of T-cell therapy or other types of cell therapy. The harvested CTLs will have high tumor specificity with a wide range of heterogeneous tumor associated antigens (TAA) presentation.

### STATEMENT REGARDING FEDERALLY SPONSORED RESEARCH OR DEVELOPMENT

This invention has been created without the sponsorship or funding of any federally sponsored research or development program.

### FIELD OF INVENTION

The present invention relates generally to a radiation oncology treatment method by way of in-situ radiation primed T-cell therapy.

### BACKGROUND OF THE INVENTION

Immunotherapy in its various forms has emerged as one of the most promising treatment modalities for cancer. Among immunotherapies, adoptive T-cell Therapy (ACT) has been recognized as the most potent and capable of achieving durable complete tumor response. Currently there are three types of T-cell therapies: Tumor Infiltrating Lymphocyte (TIL) therapy, WIC-restricted TCR gene therapy and non-WIC-restricted CAR-T therapy, where WIC is an acronym for major histocompatibility complex; TCR is an acronym for T-Cell Receptor; and CAR is an acronym for Chimeric Antigen Receptors (also known as chimeric immunoreceptors and chimeric T-cell receptors). Although impressive and encouraging early success stories abound, limitations in terms of applicability to diverse cancer types, various autoimmune toxicities and most importantly the desire of further improving tumor response rates have prompted ever more vigorous efforts in the field of immunotherapy.

In the recent decades, radiation therapy has been found to be synergistic with immunotherapy. Most appealingly, the localized radiation, especially ablative radiation, has demonstrated distal or systemic effects of inducing tumor regression—an effect sometimes referred to as the abscopal effect. The word “abscopal” means “away from target” and is used here to describe the shrinkage of untreated tumors at the same time as the shrinkage of tumors which are the target of direct or localized treatment. Studies have suggested that one of the most important pathways of this radiation-induced abscopal effect could be the adaptive immune priming pathway, whereby dying tumor cells release tumor associated antigens (TAA) that are taken up by dendritic cells and are cross-presented with MHC molecules to T-cells, such as cytotoxic T lymphocytes (CD8 or CTL or other cytotoxic T-cells), which are now primed and activated. Such an approach is generally referred to as radiation in-situ vaccination. Although immunogenic effect from radiation treatment has been evident, the robustness and extent of abscopal effects has not been consistently observed in routine clinical practice. The reasons for this could be several fold, including but not limited to: that the quantities of activated CTLs might be insufficient; and/or that the tumor and its microenvironment might be suppressive to the activated CTLs. This leads to the motivation for augmenting the radiation-induced immunogenicity in order to maximize or boost the therapeutic gain, and has resulted in a number of novel strategies. These novel strategies include, but are not limited to combining ablative radiation therapy with immune checkpoint blockades, such as anti-PD1 and anti-CTLA4. However, although effective at times, the overall therapeutic gain of current strategies to augment radiation-induced immunogenicity has not being consistently satisfactory or optimal.

### SUMMARY OF THE INVENTION

The present invention is directed towards in-situ radiation primed T-cell therapy as a method to consistently augment radiation-induced immunogenicity for optimal therapeutic gain.

The aim of this Invention is to combine the radiation-induced immunogenic effect with current T-cell therapy technique to markedly improve the therapeutic effectiveness of adoptive T-cell therapy with minimized toxicity. An essential part of this invention proposes to directly isolate from a tumor, the APC (antigen presenting cell) primed CTLs that are induced by radiation in-situ, as the source of T-cell therapy. Such CTLs will have high tumor specificity with a wide range of heterogeneous TAA presentation.

### BRIEF DESCRIPTION OF THE DRAWINGS

In describing the invention, reference will at times be made to the accompanying drawings in which:

FIG. 1 is a flow diagram illustrating the steps of the method and process of the present invention.

### DESCRIPTION OF THE INVENTION

Before the subject invention is described further, it is to be understood that the invention is not limited to the particular embodiments of the invention described below, as variations of the particular embodiments may be made and still fall within the scope of the invention. It is also to be understood that the terminology employed is for the purpose of describing particular embodiments, and is not intended to be limiting.

The details of one or more embodiments of the invention are set forth in the accompanying drawings and the description below. Other features, objects, and advantages of the invention will be apparent from the description and drawings, and from the claims. In the following description, numerous specific details are set forth to provide a thorough understanding of the embodiments. One skilled in the art to which this invention belongs will recognize, however, that the techniques described can be practiced without one or more of the specific details, or with other methods, components, materials, etc. In other instances, well known structures, materials or operations are not shown or described in detail to avoid obscuring certain aspects.

In this specification, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

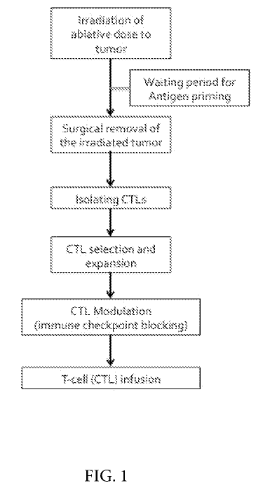

As shown in FIG. 1, the method of this invention comprises, first, identifying and examining the target tumor of a patient, then irradiating the patient's target tumor by pre-operative ablative radiation treatment. Pre-operative ablative radiation treatment may be accomplished by a variety of methods known to one of ordinary skill in the art to which this invention belongs. Then waiting for a period of time for the antigen priming of the T-cells outside of the tumor (in the lymph nodes). After a certain waiting period post-irradiation, allowing for adequate immune response and the recruitment of primed CTLs, the tumor is resected and the CTLs are harvested and isolated from the resected tumor and the CTLs undergo ex-vivo selection and expansion and modulation by subsequent treatment of immune checkpoint blockades such as anti-PD1. The waiting period post-irradiation may extend for days, but will vary depending on the tumor type, location, and other patient conditions. The expanded and modulated CTLs of therapeutic quantities will then be infused back into the patient by infusion means known to one of ordinary skill in the art to which this invention belongs. This technique as described and practiced is referred to as In-Situ Radiation-Primed T-cell Therapy (ISPT).

The therapy by this invention may be delivered for systemic treatment of microscopic disease following the removal of the primary tumor with curative intent. It may also be used with curative or palliative intent for treating advanced cancers, whereby, one of the metastatic tumors could be irradiated, resected and used as the source of in-situ primed T-cells which are isolated/harvested, expanded/modulated and then infused back into the patient to shrink and/or eliminate other tumors, wherever located in the patient's body.

T-cell therapy as prescribed by this invention may be administered as the sole treatment modality or in combination with other synergetic adjuvant therapies to further enhance treatment outcomes.

The harvested primed T-cell can also be used as the source of other tumor antigen-specific cell therapies.

Radiation is used as the stimulus for inducing immunogenicity in this invention application. Other physical means such as ultrasound, electroporation, hyperthermia, microwave/radiofrequency/cryo-ablation, radioisotope treatment, tumor treating electrical fields (Novocure), or chemical agents can also serve as stimuli for in-situ induction of immunogenicity, and may be used as an alternative stimuli in this invention.

As various changes can be made in the above-described subject matter without departing from the scope and the spirit of the invention, it is intended that all subject matter contained in the above description, shown in the accompanying drawings, or defined in the appended claims will be interpreted as descriptive and illustrative, and not in a limiting sense. Many modifications and variations of the present invention are possible in light of the above teachings. It is therefore to be understood that within the scope of the appended claims, the invention may be practiced otherwise than as specifically described.

### EQUIVALENTS

Those skilled in the art will recognize, or be able to ascertain using no more than routine experimentation, many equivalents to the specific embodiments of the invention described herein. Such equivalents are intended to be encompassed by the claims.

What is claimed is:

1. A method for in-situ radiation primed T-cell harvesting, comprising:
identifying and examining a patient with a target tumor for treatment;
irradiating the target tumor by pre-operative ablative radiation treatment;
waiting for a period of time for antigen priming of cytotoxic T-cells (CTLs) outside of the target tumor;
allowing for the build up of CTLs; then
resecting the target tumor from the patient; and then
harvesting and isolating the CTLs from the target tumor, which CTLs then undergo ex-vivo selection and expansion, followed by the modulation of immune checkpoint blockades, such as anti-PD1.

2. The method as described in claim 1, whereby the expanded and modulated CTLs are infused back into the patient for systemic treatment of microscopic disease.

3. The method as described in claim 1, whereby the expanded and modulated CTLs are infused back into the patient for systemic treatment of metastatic tumors.
